(19) United States
(12) Patent Application Publication
(10) Pub. No.: US 2014/0319383 A1
(43) Pub. Date: Oct. 30, 2014

(54) CHARGED PARTICLE BEAM IRRADIATION SYSTEM

(71) Applicant: SUMITOMO HEAVY INDUSTRIES, LTD., Tokyo (JP)

(72) Inventor: Satoru YAJIMA, Niihama-shi (JP)

(21) Appl. No.: 14/330,356

(22) Filed: Jul. 14, 2014

Related U.S. Application Data
(63) Continuation of application No. PCT/JP2012/082808, filed on Dec. 18, 2012.

(30) Foreign Application Priority Data
Jan. 16, 2012 (JP) 2012-006502

(51) Int. Cl. A61N 5/10; G21K 5/08

(52) U.S. Cl. CPC A61N 5/1078; G21K 5/08
USPC 250/492.3

(57) ABSTRACT

A charged particle beam irradiation system including: an accelerator configured to accelerate charged particles and emit a charged particle beam; a stand in which an irradiation section is disposed; a transport line which has an energy selection system configured to extract a charged particle beam having a second energy width smaller than a first energy width from a charged particle beam having the first energy width, emitted from the accelerator, and is configured to transport the charged particle beam from the accelerator to the irradiation section; and a building having an irradiation chamber and a separate room, wherein the accelerator is disposed in the irradiation chamber, the energy selection system of the transport line is disposed in the separate room, and the building has a partition wall which separates the irradiation chamber and the separate room and is configured to shield against radiation.

### INCORPORATION BY REFERENCE

Priority is claimed to Japanese Patent Application No. 2012-006502, filed Jan. 16, 2012, and International Patent Application No. PCT/JP2012/082808, the entire content of each of which is incorporated herein by reference.

### BACKGROUND

1. Technical Field

The present invention relates to a charged particle beam irradiation system for irradiating an irradiated body with a charged particle beam.

2. Description of the Related Art

As a charged particle beam irradiation system which is used for radiation therapy for cancer or the like, a charged particle beam irradiation system described in, for example, PTL 1 is known. In the charged particle beam irradiation system described in related art, in order to avoid the influence of extra radiation on a patient under treatment, a cyclotron or the majority of a transport line is disposed outside a treatment room. Further, an energy selection system (ESS) for extracting (selecting) a charged particle beam having a predetermined second energy width (an energy width smaller than a first energy width) from a charged particle beam having a predetermined first energy width emitted from a cyclotron, of a transport line from which radiation is emitted, is known.

### SUMMARY

According to an embodiment of the present invention, there is provided a charged particle beam irradiation system including: an accelerator configured to accelerate charged particles and emit a charged particle beam; a stand in which an irradiation section configured to irradiate the charged particle beam to an irradiated body is disposed; a transport line which has an energy selection system configured to extract a charged particle beam having a second energy width smaller than a first energy width from a charged particle beam having the first energy width, emitted from the accelerator, and is configured to transport the charged particle beam from the accelerator to the irradiation section; and a building having an irradiation chamber in which the stand is disposed, and a separate room in which a portion of the transport line is disposed, wherein the accelerator is disposed in the irradiation chamber, the energy selection system of the transport line is disposed in the separate room, and the building has a partition wall which separates the irradiation chamber and the separate room and is configured to shield against radiation that is emitted from the energy selection system.

### BRIEF DESCRIPTION OF THE DRAWINGS

FIG. 1 is a perspective view showing an embodiment of a charged particle beam irradiation system according to the present invention.

FIG. 2 is a plan view showing a state where a gantry in FIG. 1 is rotated by 90°.

### DETAILED DESCRIPTION

Incidentally, in the charged particle beam irradiation system described above, a reduction in size has been strongly desired in order to reduce the size of a site area. Further, in order to prevent radiation that is emitted from the energy selection system from adversely affecting a patient, it is necessary to take appropriate measures.

Therefore, it is desirable to provide a charged particle beam irradiation system in which it is possible to reduce the size of a site area while properly shielding against radiation that is emitted from an energy selection system.

According to the charged particle beam irradiation system described above, the energy selection system is disposed in the separate room separated from the irradiation chamber by the partition wall, and thus it is possible to suppress the radiation which is emitted from the energy selection system from leaking into the irradiation chamber and to properly shield against the radiation by utilizing the partition wall. In addition, in the charged particle beam irradiation system described above, the accelerator is disposed in the irradiation chamber, whereby it is possible to effectively use an extra space in the irradiation chamber and it is possible to reduce the area of the separate room in which the accelerator is disposed in a system of the related art, and therefore, it is possible to reduce the size of the site area of the entire system.

In the charged particle beam irradiation system described above, the separate room in which the energy selection system is disposed may be located on the back side of the stand.

According to this configuration, in many cases, the accelerator is disposed in an extra space on the back side of the stand, and thus it is possible to shorten the distance from the accelerator to the energy selection system, and therefore, it is advantageous for a reduction in site area.

In the charged particle beam irradiation system described above, a shielding member configured to shield against radiation heading toward the stand from the accelerator may be provided between the stand and the accelerator.

According to this configuration, it is possible to properly shield against extra radiation which heads toward a patient or the like in the stand from the accelerator in the irradiation chamber.

The stand may be a gantry configured to rotate or oscillate around a central axis line, and the stand may be disposed such that a pair of side walls facing each other with the stand interposed therebetween in the irradiation chamber is substantially parallel to the central axis line.

According to this configuration, in the structure of the gantry, an extra space of an appropriate size is formed in a corner of the irradiation chamber sandwiched between the side walls, and thus it is possible to effectively achieve a reduction in site area by disposing the accelerator therein.

According to the embodiment of the present invention, it is possible to reduce the size of a site area while properly shielding against radiation which heads toward the irradiated body from the energy selection system.

Hereinafter, a preferred embodiment of a charged particle beam irradiation system according to the embodiment of the present invention will be described in detail with reference to the drawings.

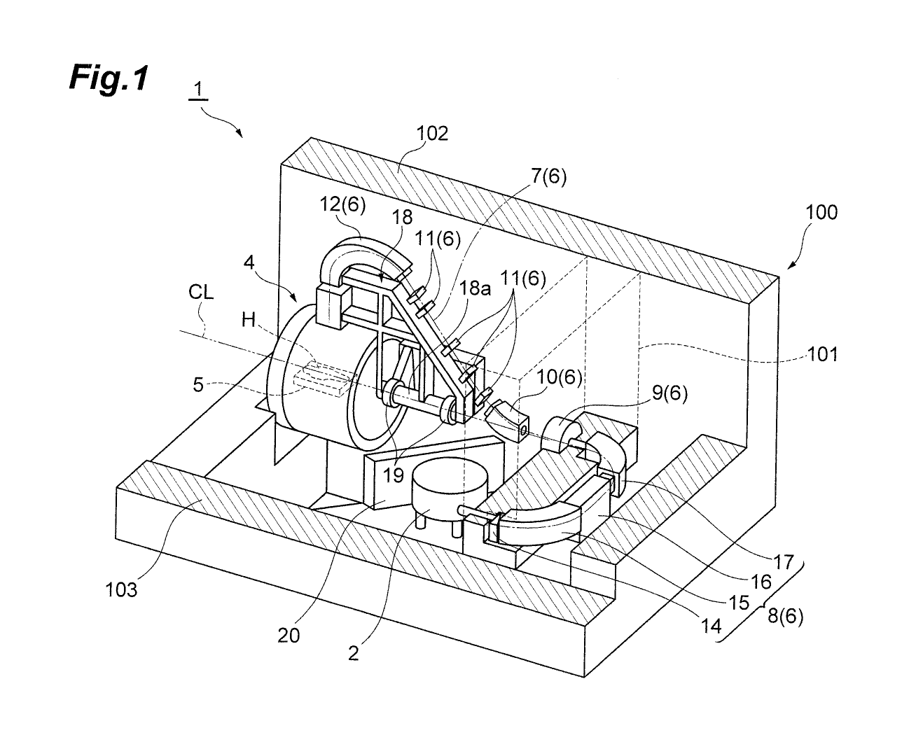

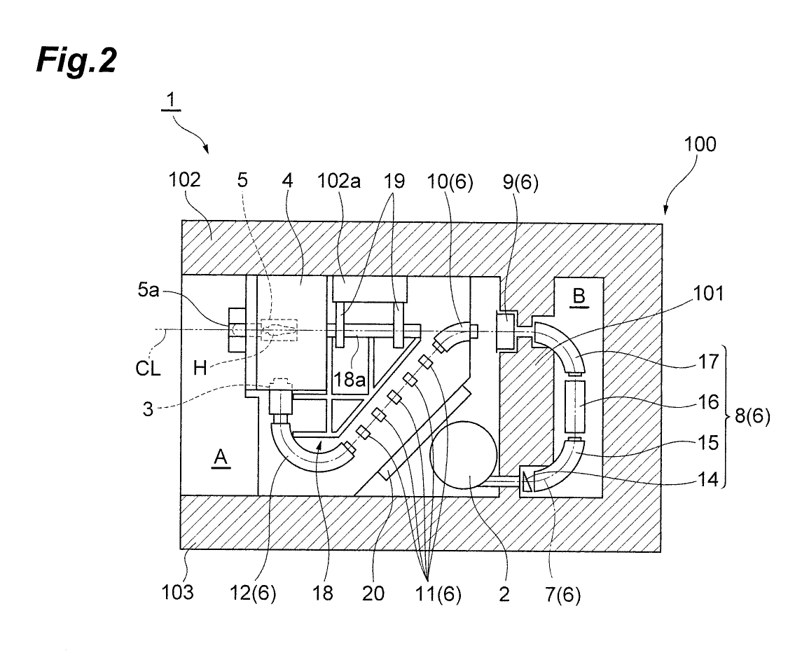

As shown in FIGS. 1 and 2, a charged particle beam irradiation system 1 according to the embodiment above is a charged particle beam irradiation system (a charged particle beam therapy system) which performs radiation therapy by irradiating a charged particle beam with respect to a tumor or the like (an irradiated body) of a patient H.

The charged particle beam irradiation system 1 is provided with an accelerator 2 which accelerates charged particles and emits a charged particle beam, a gantry (a stand) 4 in which an irradiation section 3 for irradiating the charged particle beam toward the tumor of the patient H is disposed, a treatment table 5 on which the patient is disposed, a transport line 6 which transports the charged particle beam emitted from the accelerator 2 to the irradiation section 3, and a building 100. The accelerator 2, the gantry 4, the treatment table 5, and the transport line 6 are provided in the building 100.

The building 100 is provided with an irradiation chamber A and a transport chamber (a separate room) B separated by a partition wall 101. The accelerator 2 and the gantry 4 are disposed in the irradiation chamber A and a portion of the transport line 6 is disposed in the transport chamber B. The transport chamber B is located on the back side of the gantry 4 (the opposite side to the treatment table 5). The irradiation chamber A and the transport chamber B are covered by a shielding wall which shields against radiation. In addition, in FIG. 2, illustration of the shielding wall on the left side in the paper surface of the irradiation chamber A is omitted. The partition wall 101 is formed of a material which shields against the radiation.

The accelerator 2 is a device for emitting, for example, a proton beam, a heavy particle (heavy ion) beam, or the like as the charged particle beam. As the accelerator 2, for example, a cyclotron, a synchrotron, a synchrocyclotron, a linear accelerator, or the like can be used. In order to reduce a size, it is particularly preferable to adopt a superconducting cyclotron.

The gantry 4 having a tubular shape is configured so as to be able to oscillate around a central axis line CL. The gantry 4 is disposed such that left and right side walls 102 and 103 are substantially parallel to the central axis line CL, with respect to the irradiation chamber A having a rectangular shape when viewed from above. In other words, the gantry 4 is disposed such that a pair of side walls 102 and 103 facing each other with the gantry 4 interposed therebetween extends along the central axis line CL. In front of the gantry 4, the treatment table 5 on which the patient is disposed is provided.

The treatment table 5 on which the patient is disposed is movably supported by a robot arm 5a. The robot arm 5a moves the treatment table 5 in a horizontal direction and a vertical direction.

The transport line 6 is provided with a vacuum duct 7 which connects the accelerator 2 and the irradiation section 3 of the gantry 4. In the following description, description will be made with the accelerator 2 side of the vacuum duct 7 as an upstream side and the irradiation section 3 side of the vacuum duct 7 as a downstream side.

Further, the transport line 6 is provided with an energy selection system (ESS) 8, a bearing section 9, a first deflection magnet 10, a convergence magnet 11, and a second deflection magnet 12. The constituent elements 8 to 12 are disposed to be arranged from the upstream side to the downstream side of the vacuum duct 7 in the order described above. The vacuum duct 7 is formed to pass through the partition wall 101 and to transport the charged particle beam emitted from the accelerator 2 of the irradiation chamber A to the energy selection system 8 of the transport chamber B.

The energy selection system 8 has a function to extract (select) a charged particle beam having a predetermined second energy width (an energy width smaller than a first energy width) from a charged particle beam having a predetermined first energy width emitted from the accelerator 2. The energy selection system 8 selects the energy width of the charged particle beam that the transport line 6 transports according to a treatment plan. The entirety of the energy selection system 8 is accommodated in the transport chamber B.

Specifically, the energy selection system 8 has a degrader 14, an upstream-side deflection magnet 15, an energy adjustment section 16, and a downstream-side deflection magnet 17. The degrader 14 is for attenuating (reducing) the energy of the charged particle beam emitted from the accelerator 2 and is made such that an attenuation amount (reduction amount) of energy is adjustable. The upstream-side deflection magnet 15 deflects the charged particle beam which is emitted from the accelerator 2 and in which energy is attenuated by the degrader 14, by 90° in the horizontal plane. The charged particle beam deflected by the upstream-side deflection magnet 15 proceeds toward the energy adjustment section 16.

The energy adjustment section 16 is provided with a slit for selecting the energy of the charged particle beam. When the charged particle beam having the predetermined first energy width is deflected by the upstream-side deflection magnet 15, the charged particles with low energy are deflected greatly and the charged particles with high energy are bent slightly. For this reason, the charged particle beam passes through different positions in the slit of the energy adjustment section 16 according to the energy thereof. By blocking the hole of the slit through which the charged particle beam having energy that is desired to be removed, passes, the charged particle beam having energy that is desired to be removed does not pass through the energy adjustment section 16, and only the charged particle beam having energy that is desired to be extracted passes through the energy adjustment section 16. In this way, the charged particle beam having the predetermined second energy width smaller than the first energy width can be extracted (selected) from the charged particle beam having the predetermined first energy width.

The downstream-side deflection magnet 17 deflects the charged particle beam by 90° in the horizontal plane again, thereby allowing the charged particle beam to proceed in the original direction (a direction parallel to an emission direction of the accelerator 2). In addition, a traveling direction of the charged particle beam deflected by the downstream-side deflection magnet 17 is approximately equal to an extension direction of the central axis line CL. The charged particle beam deflected by the downstream-side deflection magnet 17 passes through the partition wall 101 and proceeds to the bearing section 9.

The bearing section 9 is a site which rotatably supports the transport line 6 in the irradiation chamber A. The bearing section 9 is embedded on the irradiation chamber A side of the partition wall 101. The bearing section 9 supports the transport line 6 further along the downstream side than the bearing section 9 so as to be able to rotate around the central axis line CL.

The first deflection magnet 10 is disposed on the downstream side of the bearing section 9 and deflects the charged particle beam proceeding along the central axis line CL, in a direction away from the central axis line CL. Five convergence magnets 11 are disposed on the downstream side of the first deflection magnet 10. The convergence magnet 11 is an electromagnet which suppresses the diffusion of the charged particle beam in a radial direction of the beam.

The second deflection magnet 12 is disposed on the downstream side of the five convergence magnets 11. The second deflection magnet 12 deflects the charged particle beam toward the central axis line CL. The charged particle beam deflected by the second deflection magnet 12 proceeds toward the irradiation section 3 installed in the gantry 4.

The transport line 6 in the irradiation chamber A described above (the transport line 6 further along the downstream side than the bearing section 9) is supported by a frame 18 of the gantry 4. The frame 18 of the gantry 4 has a rotary shaft portion 18a extending along the central axis line CL and is configured so as to be able to oscillate with the rotary shaft portion 18a as the center. Specifically, the rotary shaft portion 18a is supported so as to be able to oscillate by two frame supporting sections 19 and the two frame supporting sections 19 are fixed to a convex portion 102a of the side wall 102.

The gantry 4 is supported by the frame 18 so as to be able to oscillate around the central axis line CL. Further, a trench (a hole) into which the transport line 6 enters in accordance with the oscillation of the gantry 4 is formed at the position corresponding to the lower side of the gantry 4 of the building 100. The gantry 4 oscillates in an angular range of, for example, −90° to +90° on the basis of a state where the transport line 6 is the horizontal (a state shown in FIG. 2).

Further, in order to expand the angular range in which the gantry 4 oscillates, a cut may be provided in the side wall 102. The transport line 6 enters the cut of the side wall 102, whereby it is possible to oscillate the gantry 4 in an angular range of, for example, −90° to +120°. In this manner, the gantry 4 is configured so as to oscillate within a predetermined angle range without rotating 360°, whereby it is possible to reduce a site area, compared to a case where the gantry 4 rotates 360°, and thus it is possible to attain a reduction in cost of the charged particle beam irradiation system 1.

Further, a shielding member 20 for shielding against the radiation is provided between the gantry 4 and the accelerator 2. The wall-shaped shielding member 20 is configured to include, for example, lead or the like and is formed so as to cover the gantry 4 side of the accelerator 2. The accelerator 2 in the embodiment is disposed in a space surrounded by the wall-shaped shielding member 20, the side wall 103 of the irradiation chamber A, and the partition wall 101.

The wall-shaped shielding member 20 need not extend to a ceiling and need also not be connected to the side wall 103 or the partition wall 101. It is favorable if the shielding member 20 has a configuration to properly shield against the radiation which heads toward the patient H disposed in the gantry 4 from the accelerator 2.

According to the charged particle beam irradiation system 1 related to the embodiment described above, the energy selection system 8 is disposed in the transport chamber B separated from the irradiation chamber A by the partition wall 101, and thus it is possible to suppress the radiation which is emitted from the energy selection system 8 from leaking into the irradiation chamber A and to properly shield against the radiation by utilizing the partition wall 101. As described above, the energy adjustment section 16 of the energy selection system 8 blocks the position of the hole of the slit through which the charged particle beam having energy that is desired to be removed, passes, thereby making the charged particle beam having energy that is desired to be removed collide with a member blocking the hole of the slit. If the charged particle beam having high energy from being accelerated by the accelerator 2 collides with the member blocking the hole of the slit, high energy radiation (gamma rays or the like) is generated. By shielding against the radiation having high energy by utilizing the partition wall 101, it is possible to suppress adverse effects of the radiation which is emitted from the energy selection system 8 to the patient H or the like in the irradiation chamber A. In addition, in the charged particle beam irradiation system 1, the accelerator 2 is disposed in the irradiation chamber A, whereby it is possible to effectively use an extra space in the irradiation chamber A and it is possible to reduce the area of the transport chamber B in which the accelerator 2 is disposed in a system of the related art, and therefore, it is possible to reduce the size of the site area of the entire system. Therefore, according to the charged particle beam irradiation system 1, it is possible to achieve a significant reduction in construction costs due to a reduction in site area.

Further, according to the charged particle beam irradiation system 1, the transport chamber B with the energy selection system 8 disposed therein is located on the back side of the gantry 4, whereby it is possible to shorten the distance from the accelerator 2 disposed on the back side of the gantry 4 to the energy selection system 8, and therefore, it is advantageous for a reduction in site area.

In addition, according to the charged particle beam irradiation system 1, the shielding member 20 which shields against the radiation heading toward the gantry 4 from the accelerator 2 is provided, and thus it is possible to properly shield against extra radiation which heads toward the patient or the like in the gantry 4 from the accelerator 2 in the irradiation chamber A.

Further, in the charged particle beam irradiation system 1, the gantry 4 is disposed in the irradiation chamber A such that the pair of side walls 102 and 103 facing each other with the gantry 4 interposed therebetween in the irradiation chamber A is substantially parallel to the central axis line CL. In this way, since an extra space having an appropriate size is formed in a corner of the irradiation chamber A sandwiched between the side walls 102 and 103, it is possible to effectively achieve a reduction in site area by disposing the accelerator 2 in the irradiation chamber A.

The present invention is not limited to the embodiment described above. For example, the shape of the irradiation chamber A or the transport chamber B is not limited to that described above, and various shapes can be adopted according to the installation conditions of facilities. Further, the position of the accelerator 2 or the energy selection system 8 is also not limited to that described above. For example, the accelerator 2 may be disposed at a position which is different in height from the gantry 4. In this case, it is also possible to provide the energy selection system 8 of the transport chamber B so as to extend in the vertical direction.

Further, a gantry capable of being rotated or oscillated need not to be necessarily provided, and it is possible to apply the embodiment of the present invention to even a type in which the stand with the irradiation section 3 disposed therein is fixed (a so-called fixed irradiation type). In addition, the position or the shape of the shielding member 20 is not limited to that described above, and an aspect capable of shielding against the radiation which heads toward a patient from the accelerator 2 is also acceptable.

The embodiment of the present invention is applicable to a charged particle beam irradiation system in which it is possible to reduce the size of a site area while properly shielding against radiation that is emitted from an energy selection system.

It should be understood that the invention is not limited to the above-described embodiment, but may be modified into various forms on the basis of the spirit of the invention. Additionally, the modifications are included in the scope of the invention.

What is claimed is:

1. A charged particle beam irradiation system comprising:
an accelerator configured to accelerate charged particles and emit a charged particle beam;
a stand in which an irradiation section configured to irradiate the charged particle beam to an irradiated body is disposed;
a transport line which has an energy selection system configured to extract a charged particle beam having a second energy width smaller than a first energy width from a charged particle beam having the first energy width, emitted from the accelerator, and is configured to transport the charged particle beam from the accelerator to the irradiation section; and
a building having an irradiation chamber in which the stand is disposed, and a separate room in which a portion of the transport line is disposed,
wherein the accelerator is disposed in the irradiation chamber,
the energy selection system of the transport line is disposed in the separate room, and
the building has a partition wall which separates the irradiation chamber and the separate room and is configured to shield against radiation that is emitted from the energy selection system.

2. The charged particle beam irradiation system according to claim 1, wherein the separate room in which the energy selection system is disposed is located on the back side of the stand.

3. The charged particle beam irradiation system according to claim 1, wherein a shielding member configured to shield against radiation heading toward the stand from the accelerator is provided between the stand and the accelerator.

4. The charged particle beam irradiation system according to claim 1, wherein the stand is a gantry configured to rotate or oscillate around a central axis line, and
the stand is disposed such that a pair of side walls facing each other with the stand interposed therebetween in the irradiation chamber is substantially parallel to the central axis line.
